(19) United States
(12) Patent Application Publication
(10) Pub. No.: US 2006/0153781 A1
(43) Pub. Date: Jul. 13, 2006

(54) Repair of dna mutagenic damage

(75) Inventors: Graham Edmund Kelly, Northbridge, New South Wales (AU); Alan Husband, New South Wales (AU); Cath Walker, New South Wales (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Novogen Research Pty Ltd, a Corporation, North Ryde (AU)

(21) Appl. No.: 10/526,830

(22) PCT Filed: Sep. 5, 2003
(86) PCT No.: PCT/AU03/01152

§ 371 Date: Feb. 6, 2006

(30) Foreign Application Priority Data
Sep. 6, 2002 (AU) 2002951271

(51) Int. Cl. A61K 8/49

(52) U.S. Cl. 424/59

(57) ABSTRACT

Methods for protecting skin from UV-induced DNA mutagenic damage comprising administration of one or more of equol, dehydroequol, isoflav-3-ene and isoflavan compounds in admixture with a dermally acceptable carrier are described. Also described are methods for preventing skin cancer formation.

The present invention relates to the use of equol, dehydroequol and isoflav-3-ene and isoflavan compounds in promoting repair of DNA mutagenic damage.

Metallothioneins (MT) are proteins synthesised or over expressed in response to DNA damaging agents e.g. UVR (Hansen et al 1997). In most of the studies in animals and tissue cultures, high does of radiation were used to induce MT, and therefore, it is difficult to extrapolate these results to low level or repeated exposures to UVR in humans (Cai et al 1999). Induced synthesis of MT is considered as one of the mechanisms involved in the adaptive response to low dose UVR exposure, and increased levels of MT appear to be associated with protection from UVR, possibly mediated through scavenging of ROS in the skin (Hanada, et al 1992). As well, MT is implicated in protecting against the immunosuppressive effects of UVR on cell-mediated responses as demonstrated in MT=I and II knockout mice (Reeve, et al 2000). UVR induces immunohistochemically detectable MT in keratinocytes and dermal fibroblasts concurrently with the photoconduction of p53, which suggests the these protein systems are protective and complimentary in function. MT is detectable in dermal fibroblasts from 2 hours post-UV (Anstey, et al 1996).

Equol, dehydroequol, isofla-3-ene and isoflavan compounds and methods for producing the same are described in copending International Patent Application PCT/AU03/00427 and WO 98/08503 which are incorporated herein by reference.

UV exposed skin causes damage in DNA which may give rise to carcinogenesis. The most common tumour in humans is the basal cell carcinoma (B3CC) followed by squamous cell carcinoma (SCC), and more rarely malignant melanoma.

It has now been found by the applicant that compounds of the present invention, when applied to the skin, result in elevation of metallothioneins production in the skin, particularly the basal layer of irradiated skin.

As mentioned above, metallothioneins affect and promote repair of DNA mutagenic damage of skin subject to UV exposure, and/or enhancing defence against UV-induced DNA mutagenic damage in skin.

In accordance with the present invention there is provided use of equol, dehydroequol, isoflav-3-ene or isoflavan structures for protecting skin from DNA mutagenic damage associated with UV exposure.

In another aspect there is provided use of equol, dehydroequol, isoflav-3-ene or isoflavan structures for the over expression of metallothioneins in the skin, particularly the basal layer of skin.

In accordance with another aspect of this invention there is provided a method for protecting skin from UV induced DNA mutagenic damage which comprises applying to skin a composition containing one or more of equol, dehydroequol, isoflav-3-ene, or isoflavan compounds in admixture with a dermally acceptable carrier.

Isoflav-3-ene and isoflavan compounds may be represented by the general formula (II)

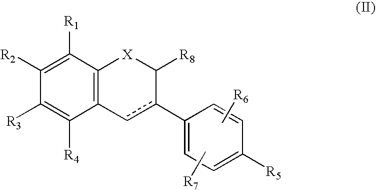

in which

R₁, R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or

R₃ and R₄ are as previously defined, and R₁ and R₂ taken together with the carbon atoms to which they are attached form a five-membered ring selected from

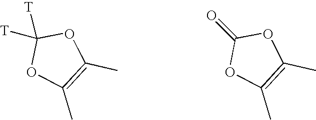

R₁ and R₄ are as previously defined, and R₂ and R₃ taken together with the carbon atoms to which they are attached form a five-membered ring selected from

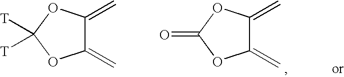

R₁ and R₂ are as previously defined, and R₃ and R₄ taken together with the carbon atoms to which they are attached form a five-membered ring selected from

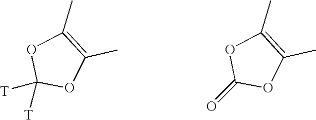

and

wherein

R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,

R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,

R₉ is alkyl, haloalkyl, aryl, arylalkyl, C(O)R₁₃ where R₁₃ is as previously defined, or Si(R₁₅)₃ where each R₁₅ is independently hydrogen, alkyl or aryl,

R₁₀ is hydrogen, alkyl, haloalkyl, amino, aryl, arylalkyl, an amino acid, alkylamino or dialkylamino,

R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,

R₁₂ is hydrogen, alkyl or aryl, or

R₁₁ and R₁₂ taken together with the nitrogen to which they are attached comprise pyrrolidinyl or piperidinyl,

the drawing [symbol] represents either a single bond or a double bond, preferably a double bond,

T is independently hydrogen, alkyl or aryl, and

X is O, NR₁₂ or S, preferably O,

including pharmaceutically acceptable salts and derivatives thereof.

Equol corresponds to the formula (II) when R₁, R₂, R₃, R₄, R₆, R₇ and R₈ are hydrogen, R₅ is hydroxy, X is O, and [symbol] is a single bond. Dehydroequol corresponds to formula (II) when R₁, R₂, R₃, R₄, R₆, R₇ and R₈ are hydrogen, R₅ is hydroxy, X is O and [symbol] is a double bond.

Dermally acceptable carriers and lotions are well known in the art, and are described for example in Remington's Pharmaceutical Sciences, Gennaro A. 18th Ed., Mack Publishing Co., Easton, Pa., 1990, pp. 1492-1517. Any dermatologically acceptable carrier can be used in the compositions of the invention. As used herein, “dermatologically acceptable carrier” refers to vehicles, diluents, carriers, which can include adjuvants, additives, or excipients, known for use in dermatological compositions. The- compositions of the invention include, but are not limited to, creams, ointments, solutions, sticks, wipes, cleansers and/or gels. The compounds of the present invention may be simply mixed, admixed or blended with suitable carriers to give compositions suitable for application to the skin. Dermally acceptable carriers may include one or more sunscreen agents. Sunscreens include those materials commonly used to block ultraviolet light. Illustrative compounds include the derivatives of cinmamate, PABA, and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed can vary depending upon the degree of protection desired from the sun's UV irradiation.

In a preferred embodiment one or more compounds of the formula (II) are formulated into cosmetic preparations. Examples of cosmetic formulations include creams, gels, powders, pastes, cakes and the like. Typically such cosmetics may be referred to as “make-up”, and/or foundation (typically used to provide a smooth, even appearance to skin and as a base for coloured cosmetics).

Compounds of the formula (II) may be used in the compositions in an amount from 0.001% to 100%, preferably from 0.1% to 20%, most preferably from 0.1% to 10% w/w. For example, compositions may comprise 1 μm to 500 mmol equol or other compounds of the formula (II), such as 20 μm to 400 μm. The remainder of the composition will comprise one or more dermatologically acceptable carriers and excipients as are well known in the art. One or more compounds may be utilised in the compositions, with equol and dehydroequol being particularly preferred. Compositions may be administered topically to the skin before, during and/or after sun exposure. Typically, doses of between about 1 to 500 mg per day, with doses between 2 to 100 mg per day being preferred.

In accordance with another aspects of this invention there is provided a method for the treatment, or amelioration or preventing the formation of skin cancer, such as basal cell carcinoma (BCC), squamous cell carcinoma (SCC) and malignant melanoma, which comprises applying to the skin of a subject a composition containing one or more of equol, dehydroequol, or an isoflav-3-ene or isoflavan compounds of the general formula (II).

In another aspect of this invention there is provided a method for increasing metallothionein production in the skin, such as the basal layer of skin, which comprises applying to skin one or more of equol, dehydroequol, isoflav-3-ene or isoflavan compound in association with a dermally acceptable carrier.

The applicant has further found that the compounds according to this invention promote DNA repair. The promotion of DNA repair may be by one or more of increasing the rate of repair of cyclobutane pyrimidine dimers (CPDs), promoting DNA repair by decreasing P53 expression, and/or by promoting the formation of metallothionein (MT).

The formation of CPD is considered to be an important lethal and mutagenic consequence of UVR exposure (Mitchell et al, 1989; Liardet et al, 2000). Animal models have demonstrated an inverse relationship between epidermal CPD repair and skin carcinogenesis (Young et al, 1996). The P53 protein (TP53) is expressed after DNA damage by UV irradiation. P53 is a transcription factor which blocks cellular progression from G1 to S phase, thus preventing replication of damaged DNA (Campbell et al, 1993). The P53 protein may act as a tumour promoting agent (Murphey et al, 2001).

This invention will be described with reference to the following, non-limiting examples.

### EXAMPLE 1

The effect of equol on the induction of CPD was examined in the skin of hairless mice (a standard model for human dermatological investigations) exposed to solar simulated ultraviolet radiation (SSUV). At various time points after SSUV, dorsal skin was excised, fixed for 6 hr in a standard fixing medium (HistoChoice™, Amersco Inc, Solon, Ohio, USA), processed and paraffin-embedded. Pyrimidine dimers were detected immunohistochemically using citric acid antigen retrieval and the H3 anti-pyrimidine dimer antibody. The number of dimer-positive cells was counted manually in 30 fields per mouse, at 40× magnification.

When equol lotion (containing 20 μM equol) was applied daily for 7 days prior to and following irradiation with 1×3 MED of SSUV, the effect of equol was to reduce the initial induction of dimers, and to enhance the rate of their repair, as evidenced by a reduced number of dimers at 24 hr (Table 1).

TABLE 1
Induction of epidermal CPD-positive cells following UV irradiation

| | | CPD + ve |
|---|---|---|
| Time of collection | Treatment | cells/linear cm |
| 1 hr post-SSUV | Normal skin | 0 |
| | Vehicle + SSUV | 300 ± 18 |
| | equol + SSUV | 238 ± 22 |
| 24 hr post-SSUV | Vehicle + SSUV | 340 ± 55 |
| | equol + SSUV | 167 ± 17 |

Application of equol immediately after SSUV exposure (and continuing for 5 d) resulted in significantly reduced dimers at 1 day post-irradiation (a significant reduction of 23%), and at 2 d (a significant reduction of 42% -data not shown).

When equol lotion (20 μM) was applied for both 7 days prior and 5 days after SSUV exposure, the reduction in CPD numbers was evident immediately and at 1, 24 and 48 hours after (p<0.05; 54%, 50% and 26% reduction in the number of CPD respectively) compared with the control group (vehicle alone).

### EXAMPLE 2

Equol was applied to the skin of five human volunteers immediately after, and at 4 hours and 6 hours post-UV irradiation. A control lotion was also used containing no equol. Twenty-four hours after UV irradiation, skin biopsies were taken and MT production was measured using immunohistochemistry.

Table 2 shows the counts of cells in the basal epidermis and superficial dermis that demonstrated positive staining for MT. Approximately half of the cells in the basal epidermis constitutively expressed MT at baseline, whereas almost none of the cells in the more superficial layers of the epidermis expressed MT. At 24 hrs after exposure to 2.5 MED SSUV, there were apparent differences in the expression of MT in the basal layers of the epidermis between sections treated with equol and those treated with DMSO in base lotion (vehicle). In all 5 participants, the expression of MT was higher in the skin treated with equol, with the magnitude of the difference ranging from +4% to +21%.

TABLE 2
Proportion of cells staining positively for MT in the epidermis of
five human volunteers, by treatment group

| | | Total epidermis | | | Upper epidermis | | | Basal epidermis | | |
|---|---|---|---|---|---|---|---|---|---|---|
| Subject | treatment | neg | pos | % | neg | pos | % | neg | pos | % |
| NO1DWH | Baseline | 303 | 201 | 40 | 99 | 0 | 0 | 204 | 201 | 50 |
| | 10 mins | 255 | 179 | 41 | 72 | 0 | 0 | 183 | 179 | 49 |
| | DMSO | 282 | 185 | 40 | 70 | 0 | 0 | 212 | 185 | 47 |
| | equol | 303 | 382 | 56 | 185 | 2 | 1 | 118 | 380 | 76 |
| NO3PPA | Baseline | 227 | 109 | 32 | 97 | 0 | 0 | 130 | 109 | 46 |
| | 10 mins | 231 | 237 | 51 | 77 | 4 | 5 | 154 | 233 | 60 |
| | DMSO | 317 | 236 | 43 | 96 | 4 | 4 | 221 | 232 | 51 |
| | equol | 270 | 271 | 50 | 82 | 0 | 0 | 188 | 271 | 59 |
| NO6MED | Baseline | 420 | 413 | 50 | 169 | 0 | 0 | 251 | 413 | 62 |
| | 10 mins | 437 | 565 | 56 | 168 | 1 | 1 | 269 | 564 | 68 |
| | DMSO | 440 | 442 | 50 | 130 | 6 | 4 | 310 | 436 | 58 |
| | equol | 315 | 539 | 63 | 76 | 8 | 10 | 239 | 531 | 69 |
| N13PDO | Baseline | 267 | 217 | 45 | 112 | 0 | 0 | 155 | 217 | 58 |
| | 10 mins | 468 | 703 | 60 | 270 | 10 | 4 | 198 | 693 | 78 |
| | DMSO | 465 | 405 | 47 | 144 | 0 | 0 | 321 | 405 | 56 |
| | equol | 323 | 527 | 62 | 169 | 5 | 3 | 154 | 522 | 77 |
| N14GBO | Baseline | 270 | 127 | 32 | 113 | 0 | 0 | 157 | 127 | 45 |
| | 10 mins | 381 | 242 | 39 | 247 | 0 | 0 | 134 | 242 | 64 |
| | DMSO | 276 | 217 | 44 | 111 | 4 | 3 | 165 | 213 | 56 |
| | equol | 225 | 234 | 51 | 68 | 1 | 1 | 157 | 233 | 60 |

Note:
“Baseline” refers to the skin sections from the punch biopsy taken prior to exposure to 2.5 MED SSUV.
“10 mins” refers to the skin sections from the punch biopsy taken 10 mins after exposure to 2.5 MED SSUV. The skin was not treated with either DMSO in base lotion (vehicle) or equol at 200 μM.
“DMSO” refers to the skin sections from the punch biopsy taken 24 hrs after exposure to 2.5 MED SSUV. The skin was from the grid treated with DMSO in base lotion (vehicle).
“Equol” refers to the skin sections from the punch biopsy taken 24 hrs after exposure to 2.5 MED SSUV. The skin was from the grid treated with equol at 200 μM.

The increase of MT immunoreactivity in basal and suprabasal keratinocytes of recently UV-exposed individuals was highest in skin that had been treated with equol.

### EXAMPLE 3

The skin biopsies from the five human volunteers from Example 2 were tested for cyclobutane pyrimidine dimer formation using immunohistochemistry.

Table 3 presents the counts and percentages of cells staining positively with an antibody directed against CPD. These data demonstrate that, as expected, there were essentially no CPD-positive cells in the epidermis prior to irradiation with 2.5 MED. However, skin sections taken from all of the participants 10 mins after UV exposure showed high levels of DNA damage, with the proportion of positively-staining cells ranging from 36% participants N01DWH and N03PPA) to 87% (participant N14GBO).

Skin sections taken 24 hrs after UV exposure showed substantially lower levels of CPD damage in all subjects. For 4 out of 5 participants, the skin sections treated with equol lotion had proportionally less CPD-positive cells than the skin sections treated with DMSO in base lotion (vehicle).

TABLE 3
Proportion of cells staining positively for CPDs in the epidermis
of five human volunteers, by treatment group

| | | Total epidermis | | | Upper epidermis | | | Basal epidermis | | |
|---|---|---|---|---|---|---|---|---|---|---|
| Subject | treatment | neg | pos | % | neg | pos | % | neg | pos | % |
| NO1DWH | Baseline | 345 | 0 | 0 | 134 | 0 | 0 | 211 | 0 | 0 |
| | 10 mins | 162 | 107 | 40 | 64 | 51 | 44 | 98 | 56 | 36 |
| | DMSO | 231 | 105 | 31 | 81 | 47 | 37 | 150 | 58 | 28 |
| | equol | 164 | 39 | 19 | 70 | 23 | 25 | 94 | 16 | 15 |
| NO3PPA | Baseline | 309 | 0 | 0 | 104 | 0 | 0 | 205 | 0 | 0 |
| | 10 mins | 204 | 191 | 48 | 56 | 106 | 65 | 148 | 85 | 36 |
| | DMSO | 179 | 25 | 12 | 55 | 20 | 27 | 124 | 5 | 4 |
| | equol | 349 | 18 | 5 | 70 | 17 | 20 | 279 | 1 | 0 |
| NO6MED | Baseline | 309 | 0 | 0 | 90 | 0 | 0 | 219 | 0 | 0 |
| | 10 mins | 136 | 364 | 73 | 19 | 198 | 91 | 117 | 166 | 59 |
| | DMSO | 339 | 65 | 16 | 112 | 59 | 35 | 227 | 6 | 3 |
| | equol | 279 | 92 | 25 | 98 | 71 | 42 | 181 | 21 | 10 |
| N13PDO | Baseline | 205 | 0 | 0 | 60 | 0 | 0 | 145 | 0 | 0 |
| | 10 mins | 69 | 195 | 74 | 20 | 94 | 82 | 49 | 101 | 67 |
| | DMSO | 105 | 68 | 39 | 78 | 51 | 40 | 27 | 17 | 39 |
| | equol | 213 | 94 | 31 | 79 | 50 | 39 | 134 | 44 | 25 |
| N14GBO | Baseline | 255 | 0 | 0 | 98 | 0 | 0 | 157 | 0 | 0 |
| | 10 mins | 34 | 389 | 92 | 0 | 157 | 100 | 34 | 232 | 87 |
| | DMSO | 240 | 131 | 35 | 93 | 69 | 43 | 147 | 62 | 30 |
| | equol | 188 | 85 | 31 | 63 | 44 | 41 | 125 | 41 | 25 |

Note:
“Baseline” refers to the skin sections from the punch biopsy taken prior to exposure to 2.5 MED SSUV.
“10 mins” refers to the skin sections from the punch biopsy taken 10 mins after exposure to 2.5 MED SSUV. The skin was not treated with either DMSO in base lotion (vehicle) or equol at 200 μM.
“DMSO” refers to the skin sections from the punch biopsy taken 24 hrs after exposure to 2.5 MED SSUV. The skin was from the grid treated with DMSO in base lotion (vehicle).
“Equol” refers to the skin sections from the punch biopsy taken 24 his after exposure to 2.5 MED SSUV. The skin was from the grid treated with lotion containing equol at 200 μM.

When data from all participants were pooled, it can be seen that skin sections treated with equol had moderately lower levels of CPD damage at 24 hours.

### EXAMPLE 4

The skin biopsies from the five human volunteers from Example 2 were tested for P53 staining following UV irradiation. Results are shown in Table 4.

TABLE 4
Proportion of cells staining positively for p53 in the epidermis of
five human volunteers, by treatment group

| | | Total epidermis | | | Upper epidermis | | | Basal epidermis | | |
|---|---|---|---|---|---|---|---|---|---|---|
| Subject | treatment | neg | pos | % | neg | pos | % | neg | pos | % |
| NO1DWH | Baseline | 261 | 0 | 0 | 115 | 0 | 0 | 146 | 0 | 0 |
| | 10 mins | 343 | 1 | 0 | 154 | 1 | 1 | 189 | 0 | 0 |
| | DMSO | 187 | 12 | 6 | 79 | 4 | 5 | 108 | 8 | 7 |
| | equol | 270 | 94 | 26 | 109 | 48 | 31 | 161 | 46 | 22 |
| NO3PPA | Baseline | 274 | 2 | 1 | 112 | 1 | 1 | 162 | 1 | 1 |
| | 10 mins | 316 | 2 | 1 | 114 | 2 | 2 | 202 | 0 | 0 |
| | DMSO | 223 | 55 | 20 | 81 | 31 | 28 | 142 | 24 | 14 |
| | equol | 337 | 87 | 21 | 165 | 72 | 30 | 172 | 15 | 8 |
| NO6MED | Baseline | 412 | 1 | 0 | 134 | 0 | 0 | 278 | 1 | 0 |
| | 10 mins | 402 | 3 | 1 | 153 | 1 | 1 | 249 | 2 | 1 |
| | DMSO | 462 | 133 | 22 | 165 | 77 | 32 | 297 | 56 | 16 |
| | equol | 500 | 50 | 9 | 250 | 19 | 7 | 250 | 31 | 11 |
| N13PDO | Baseline | 325 | 0 | 0 | 141 | 0 | 0 | 184 | 0 | 0 |
| | 10 mins | 304 | 0 | 0 | 140 | 0 | 0 | 164 | 0 | 0 |
| | DMSO | 222 | 45 | 17 | 109 | 8 | 7 | 113 | 37 | 25 |
| | equol | 287 | 13 | 4 | 147 | 4 | 3 | 140 | 9 | 6 |
| N14GBO | Baseline | 321 | 0 | 0 | 149 | 0 | 0 | 172 | 0 | 0 |
| | 10 mins | 292 | 4 | 1 | 185 | 2 | 1 | 107 | 2 | 2 |
| | DMSO | 217 | 190 | 47 | 106 | 106 | 50 | 111 | 84 | 43 |
| | equol | 227 | 76 | 25 | 109 | 35 | 24 | 118 | 41 | 26 |

Note:
“Baseline” refers to the skin sections from the punch biopsy taken prior to exposure to 2.5 MED SSUV.
“10 mins” refers to the skin sections from the punch biopsy taken 10 mins after exposure to 2.5 MED SSUV. The skin was not treated with either DMSO in base lotion (vehicle) or equol at 200 μM.
“DMSO” refers to the skin sections from the punch biopsy taken 24 hrs after exposure to 2.5 MED SSUV. The skin was from the grid treated with DMSO in base lotion (vehicle).
“Equol” refers to the skin sections from the punch biopsy taken 24 hrs after exposure to 2.5 MED SSUV. The skin was from the grid treated with equol at 200 μM.

As expected, there were essentially no cells in the epidermis expressing p53 prior to irradiation with 2.5 MED for any of the participants. Similarly, skin sections taken from participants 10 mins after UV exposure showed negligible levels of p53 expression, in accordance with the literature.

Skin sections taken 24 hrs after UV exposure showed substantially higher levels of p53 expression in all subjects. The percentage of p53 expression in upper and/or basal epidermis was reduced in four out of five equol treated subjects. For example, in subjects N13PDO and N14GBO the percentage of p53 staining was reduced significantly (generally more than 50%) compared with vehicle controls.

Conclusions

The biomarkers assessed in these experiments were selected based on their biological associations with skin cancer (which is directly associated with UV-induced DNA mutagenic damage).

UV-induced oxidative damage is now recognised as a potentially important causal factor in skin cancer. MTs are molecules with anti-oxidant properties that are specifically induced in response to UV exposure. This study found consistent evidence that human skin treated with equol, and it is believed other compounds of the formula (II), induce more MT than skin treated with base lotion.

CPDs are the earliest indicator of molecular damage following exposure to UV radiation, and if not repaired, lead to fixed mutations in the DNA of skin cells. Thus one mechanism of action of a post-exposure treatment would be to increase the rate of repair of these lesions. The experiments conducted here suggest that CPD repair may be enhanced by topical equol compositions, and other compositions containing one or more compounds of the formula (II).

P53 is clearly an important regulatory gene that is commonly mutated in epidermal skin cancers. Moreover, in normal skin cells, p53 is up-regulated following UV exposure to prevent mitosis until DNA damage is repaired. Equol modulated the expression of p53 in this study causing a reduction in the number of cells in the upper or basal epidermis expressing p53 for four of five subjects.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The reference to any prior art in this specification is not, and should not be taken as an acknowledgment or any form of suggestion that that prior art forms part of the common general knowledge in Australia.

### REFERENCES

Anstey, A., R. Marks, C. Long, H. Navabi. A. Pearse, D. Wynford-Thomas and B. e. Jasani (1996). “In vivo photoinduction of metallothionein in human skin by ultraviolet irradiation.” Journal of Pathology 178(1): 84-8.

Beme, B., J. Ponten and F. Ponten (1998). “Decreased p53 expression in chronically sun-exposed human skin after topical photoprotection.” Photodermatology, Photoimmunology & Photomedicine 14(5-6): 148-53.

Cai, L., M. Satoh, C. Tohyama and M. G. Cherian (1999). “Metallothionein in radiation exposure: its induction and protective role.” Toxicology 132(2-3): 85-98.

Campbell, C., A. G. Quinn, B. Angus, P. M. Farr and J. L. Rees (1993). “Wavelength specific patterns of p53 induction in human skin following exposure to UV radiation.” Cancer Research 53(12): 2697-9.

Hanada, K., T. Baba, I. Hashimoto, R. Fukui and S. Watanabe (1992). “Possible role of cutaneous metallothionein in protection against photo-oxidative stress—epidermal localization and scavenging activity for superoxide and hydroxyl radicals.” Photodermatology, Photoimmunology & Photomedicine 9(5): 209-13.

Hansen, C., E. Ablett, A. Green, R. A. Stunn, I. S. Dunn, D. P. Fairlie, M. L. West and P. G. Parsons (1997). “Biphasic response of the metallothionein promoter to ultraviolet radiation in human melanoma cells.” Photochemistry & Photobiology 65(3): 550-5.

Liardet, S., C. Scaletta, R. Panizzon. P. Hohlfeld and L. Laurent-Applegate (2001). “Protection against pyrimidine dimers, p53, and 8-hydroxy-2′-deoxyguaosine expression in ultraviolet-irradiated human skin by sunscreens: Difference between UVB+UVA and UVA alone sunscreens.” Journal of Investigative Dermatology 117:1437-1441.

Mitchell, D. L. and R. S. Nairn (1989). “The biology of the (6-4) photoproduct.” Photochemistry & Photobiology 49(6): 805-19.

Murphy, G., A. R. Young, H. C. Wulf, D. Kuhms and T. Schwarz (2001). “The molecular determinants of sunburn cell formation.” Experimental Dermatology 10(3): 155-60.

Ponten, F., B. Berne, Z. P. Ren, M. Nister and J. Ponten (1995). “Ultraviolet light induces expression of p53 and p21 in human skin: effect of sunscreen and constitutive p21 expression in skin appendages.” Journal of investigative Dermatology 105(3): 402-6.

Reeve, V. E., N. Nishimura, M. Bosnic. A. E. Michalska and K. H. Choo (2000). “Lack of metallothionein-I and -II exacerbates the immunosuppressive effect of ultraviolet B radiation and cis-urocanic acid in mice.” Immunology 100(3): 399-404.

Rich, T., R. L. Allen and A. H. Wyllie (2000). “Defying death after DNA damage.” Nature 407(6805): 777-83.

Seite, S., D. Moyal, M. P. Verdier, C. Hourseau and A. Fourtanier (2000). “Accumulated p53 protein and UVA protection level of sunscreens.” Photodermatology, Photoimmunology & Photomedicine 16(1): 3-9.

Vainio, H. and F. Bianchini, Eds. (2001). Sunscreens. LARC Handbooks of Cancer Prevention. Lyon, International Agency for Research on Cancer.

Young, A. R., C. A. Chadwick, G. I. Harrison, J. L. Hawk, O. Nikaido and C. S. Potten (1996). “The in situ repair kinetics of epidermal thymine dimers and 6-4 photoproducts in human skin types 1and II.” Journal of Investigative Dermatology 106(6): 1307-13.

1. A method for promoting repair of UV-induced, DNA mutagenic damage in skin and/or enhancing defence against UV-induced DNA mutagenic damage in skin which comprises administering topically to the skin a composition containing one or more compounds of the general formula (II): 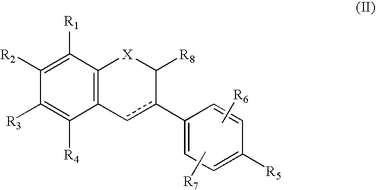
in which
R₁,R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₃ and R₄ are as previously defined, and R₁ and R₂ taken together with the carbon atoms to which they are attached form a five-membered ring selected from 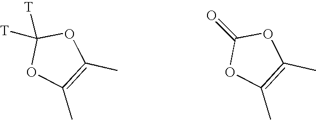
R₁ and R₂ are as previously defined, and R₃ and R₄ taken together with the carbon atoms to which they are attached form a five-membered ring selected from 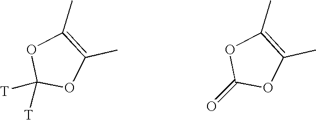
and
wherein
R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,
R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,
R₈ is alkyl, haloalkyl, aryl, arylalkyl, C(O)R₁₃ where R₁₃ is as previously defined, or Si(R₁₅)₃ where each R₁₅ is independently hydrogen, alkyl or aryl,
R₁₀ is hydrogen, alkyl, haloalkyl, amino, aryl, arylalkyl, an amino acid, alkylamino or dialkylamino,
R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,
R₁₂ is hydrogen, alkyl or aryl, or
R₁₁ and R₁₂ taken together with the nitrogen to which they are attached comprise pyrrolidinyl or piperidinyl,
the drawing [symbol] represents either a single bond or a double bond, preferably a double bond,
T is independently hydrogen, alkyl or aryl, and
X is O, NR₁₂ or S, preferably O,
including pharmaceutically acceptable salts and derivatives thereof in admixture with a dermatologically acceptable carrier.

2. A method according to claim 1 wherein said one or more compounds of the formula (II) comprise equol and dehydroequol.

3. A method according to claim 1 which is a method for preventing the formation of skin cancer.

4. A method according to claim 3 wherein skin cancer is selected from basal cell carcinoma, squamous cell carcinoma and malignant melanoma.

5. A method according to claim 1 wherein skin is protected from UV-induced mutagenic damage by one or more of increasing the rate of repair of cyclobutane pyrimidine dimers, promoting the formation of metallothionein, and decreasing p53 expression.

6. A method according to claims 1 to 5 wherein the composition is administered before, during and/or after UV exposure.

7. A method according to claim 6 wherein the composition is administered before UV exposure.

8. A method according to claim 6 wherein the composition is administered before and after UV exposure.

9. A method according to claims 1 to 8 wherein the composition comprises 20 μm to 500 mmol of compounds of the formula (II).

10. Use of one or more compounds of the formula (II) 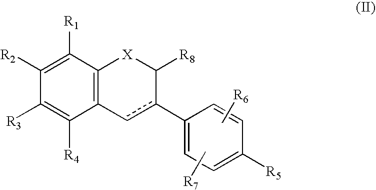
in which
R₁,R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₃ and R₄ are as previously defined, and R₁ and R₂ taken together with the carbon atoms to which they are attached form a five-membered ring selected from 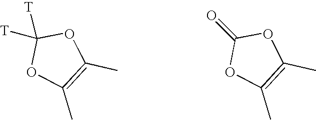
R₁ and R₄ are as previously defined, and R₂ and R₃ taken together with the carbon atoms to which they are attached form a five-membered ring selected from 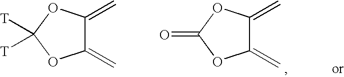
R₁ and R₂ are as previously defined, and R₃ and R₄ taken together with the carbon atoms to which they are attached form a five-membered ring selected from 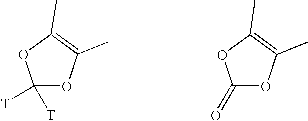
and
wherein
R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,
R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,
R₈ is alkyl, haloalkyl, aryl, arylalkyl, C(O)R₁₃ where R₁₃ is as previously defined, or Si(R₁₅)₃ where each R₁₅ is independently hydrogen, alkyl or aryl,
R₁₀ is hydrogen, alkyl, haloalkyl, amino, aryl, arylalkyl, an amino acid, alkylamino or dialkylamino,
R₁₁ is hydrogen, alkyl, arylalkyl, alkenyl, aryl, an amino acid, C(O)R₁₃ where R₁₃ is as previously defined, or CO₂R₁₄ where R₁₄ is as previously defined,
R₁₂ is hydrogen, alkyl or aryl, or
R₁₁ and R₁₂ taken together with the nitrogen to which they are attached comprise pyrrolidinyl or piperidinyl,
the drawing [symbol] represents either a single bond or a double bond, preferably a double bond,
T is independently hydrogen, alkyl or aryl, and
X is O, NR₁₂ or S, preferably O,
including pharmaceutically acceptable salts and derivatives thereof in admixture with a dermatologically acceptable carrier for the manufacture of a topical composition for promoting repair of UV-induced DNA mutagenic damage in skin, and/or enhancing defence against UV induced DNA mutagenic damage in skin.

11. Use according to claim 10 wherein said one or more compounds of the formula (II) comprise equol and dehydroequol.

12. Use according to claim 10 which is a method for preventing the formation of skin cancer.

13. Use according to claim 12 wherein skin cancer is selected from basal cell carcinoma, squamous cell carcinoma and malignant melanoma.

14. Use according to claim 10 wherein skin is protected from DNA mutagenic damage by one or more of increasing the rate of repair of cyclobutane pyrimidine dimers, promoting the formation of metallothionein, and decreasing p53 expression.

15. Use according to claims 10 to 14 wherein the composition is administered before, during and/or after UV exposure.

16. Use according to claim 15 wherein the composition is administered before UV exposure.

17. Use according to claim 15 wherein the composition is administered before and after UV exposure.

18. Use according to claims 10 to 17 wherein the composition comprises 20 μm to 500 mmol of compounds of the formula (II).

19. Use of compounds of the formula (II) for promoting repair of UV-induced DNA mutagenic damage in skin and/or enhancing defence against UV induced DNA mutagenic damage in skin.

20. A method according to any of claims 1 to 9 where the composition comprises a cosmetic or sunscreen composition.

21. A use according to claims 10 to 19 wherein the composition comprises a cosmetic or sunscreen composition.

22. A cosmetic or sunscreen composition which comprises one or more compounds of the formula (II) as hereinbefore defined in association with one or more dermally acceptable carriers or excipients.

23. A cosmetic composition according to claim 22 which comprises a make-up or foundation composition.
